(19) United States
(12) Patent Application Publication
(10) Pub. No.: US 2007/0110817 A1
(43) Pub. Date: May 17, 2007

(54) Biological active blood serum obtained by electrostimulation

(75) Inventor: Vitali A. Shestakov, Moscow (RU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Owen Holding Ltd., Douglas (GB)

(21) Appl. No.: 10/581,420

(22) PCT Filed: Dec. 20, 2004
(86) PCT No.: PCT/EP04/14510

§ 371 Date: Sep. 18, 2006

Related U.S. Application Data
(60) Provisional application No. 60/619,738, filed on Oct. 18, 2004.

(51) Int. Cl. A61K 35/16; A61K 9/68; A61K 9/127; A61K 9/48

(52) U.S. Cl. 424/531; 424/451; 424/440; 424/450

(57) ABSTRACT

The present invention relates to a method for preparing a blood serum product, the blood serum product and a pharmaceutical composition comprising said blood serum product as well as uses thereof in the treatment of various diseases and conditions, including epileptic seizures and apoplexy.

The present invention relates to a method for preparing a blood serum product, the blood serum product, and a pharmaceutical composition comprising said blood serum product as well as uses thereof in the treatment of various diseases and conditions including epileptic seizures and apoplexy.

### BACKGROUND OF THE INVENTION

Methods for the preparation of active substances from blood serum are known, in the art. One is based on the withdrawal of blood from humans or animals, the subsequent incubation as well as the separation of the active substance and finally the preservation of the substance (see, for example, JP 2123287, EP 0 542 303, RU 2096041, RU 2120301). The prior art method concerns the preparation of a blood serum which improves the resistance of the body in respect of exogenic and endogenic factors like air pressure, air temperature, gravity, light etc. as well as hunger, thirst, sleeping and sexual desires etc. The blood serum is drawn from the donor who has previously been brought into a certain functional state and according to the length of the application of the functional state and the type of functional state, e.g. sleep deprivation, alcohol abuse, nicotine abuse etc., blood serum with different biological activity can be obtained which shows mitogenic, somnogenic, opthalmogenic, audio active, therno active, diatary active, sexually active, anti-hypoxic, anti-alcohol and anti-nicotine activity.

A different method is disclosed in EP 1 283 047 and concerns the treatment of animal blood serum by gamma irradiation with the aim to increase the biological activity of the blood serum product.

Presently, a lot of research focuses on the mechanisms, which regulate cellular proliferation of different human tissues. In this respect both stimulators and inhibitors of proliferation of normal and pathologically abnormal somatic cells, including nerve cells, are investigated (see, for example Aschmarin, I. P. “Neurochemistry”, Moskwa, Publishers of the Biomedical Chemical Institute of the Russian Academy of Science”, 1996).

It has been observed that peptide growth factors apart from their general activating functions, e.g. stimulation of mitosis, cell differentiation and cell growth of different types of normal tissue, increased wound healing, can cause tumor formation and proliferation (see, for example, Bouneres, P. (1993), Horm. Res. 40: 31; Robinson, C., (1993) Ann. Med. 25: 535; Dignez, C. and Casanueva F. (1995) Trends Endocrin. Metab. 6: 55, Menster D. et al. (1995) Clin. Exp. Metastasis 13: 67).

Such peptides as, for example, the parathyroid peptides, gastrin or bombesin foster the development of tumor cells as well as the development of breast, bone and colon cancer (see, for example, Kitazawa S. and Maeda S. (1995) Clin. Orthop. 312: 45-50 and Kaji et al. (1995) Endocrinology 136: 842).

Although some peptides facilitate normal cell division and are stimulators for human and animals there is the danger that the use will lead to tumor cell development and eventually to the development of cancer.

Earlier experiments have shown that stimulation of animals with electricity leads to an increase of the P-endorphin level in the blood (see, for example, Litvinova S. V. et al. (1990) Biomed. Sci. 5: 471). In a reference work by Udovitschenko, W. I. numerous data is provided with respect to the results of stimulation or shock due to various causes. It has been shown that, for example, electroshock leads to a marked increase of the concentration of β-endorphines, meta- and leu-encephalines within the blood (see Udowitschenko, W. I. (1989) “Xenogenic Opioid System in Shock” Pathiological Physiology and Experimental Therapy” 6: 72-77).

### SUMMARY OF THE INVENTION

An object of the present invention was the development of a novel method for the preparation of a biological active blood serum from animal blood. Surprisingly it was found that the biological active blood serum prepared according to the method of the invention exhibited new therapeutic properties.

Consequently, one aspect of the present invention is a method for producing a biological active blood serum comprising the steps of:

a) electrostimulation of a non-human animal

b) withdrawal of blood from said animal,

c) isolation of serum from said blood, and

d) gamma irradiation of said serum.

In a preferred embodiment the non-human animal is selected form the group consisting of mammals and birds, preferably from poultry, e.g. chicken, dug, goose, ostrich, and quail.

Although the electrostimulation can be employed to any part of the body it is preferred that step a) of the method of the present invention is applied to the head, the neck, the body and/or one or more limbs of the animal. Out of those it is preferred that the head of the animal is electrostimulated. In the context of the present invention the terms electrostimulation and electroshock are used interchangeably.

In a preferred embodiment of the method of the present invention the electrostimulation is carried out for a time period of between 1 and 60 seconds, preferably between 1 and 30 seconds, and more preferably between 2 and 10 seconds. It is also preferred that the electrostimulation is carried out with a voltage in the range of between 50 V and 150 V, preferably in the range of between 80 V to 120 V, and more preferably in the range of between 110 V and 120 V. During the performance of the electrostimulation certain currents are preferred and preferably the electrostimulation is carried out with a current in the range of between 0.01 A and 0.4 A, preferably in the range of between 0.02 A and 0.1 A, and more preferably in the range of between 0.04 A and 0.06 A.

In a preferred embodiment of the method of the present invention the electrostimulation is carried out with a frequency in the range of between 10 and 200 Hz, preferably in the range of between 20 to 100 Hz and more preferably in the range of between 45 to 65 Hz.

In a further preferred embodiment of the method of the present invention the gamma irradiation is administered with an adsorbed radiation dose of between 10 to 40 kGy, preferably 15 to 35 kGy and more preferably of between 20 and 30 kGy. The gamma radiation source can be any source, however, a preferred source of gamma radiation is selected from the group consisting of ⁶⁰Co, ¹³⁷Cs, ⁶⁷Cu, ⁶⁷Ga, ¹¹¹In, ¹⁹²Ir, ^{99m}Tc and ¹⁷⁰Tm.

In a further preferred embodiment of the method of the present invention the method further comprises the step of incubating said blood prior to step c).

In a further preferred embodiment of the method of the present invention the method further comprises the step of lyophilization of said serum prior to step d).

In a preferred embodiment of the method of the present invention the blood is arterial and/or venous blood.

Another aspect of the present invention is the biological active blood serum which is producible according to a method of the present invention.

A further aspect of the present invention is a pharmaceutical composition comprising a blood serum according to the present invention and one or more pharmaceutically acceptable diluents; carriers; excipients, including fillers, binders, lubricants, glidants, disintegrants, adsorbents; and/or preservatives.

In a preferred embodiment of the pharmaceutical composition of the present invention the composition is formulated as a syrup, an infusion or injection solution, a tablet, a capsule, a capslet, lozenge, a liposome, a suppository, a plaster, a band-aid, a retard capsule, a powder, or a slow release formulation. Preferably the diluent is water, a buffer, a buffered salt solution or a salt solution and the carrier preferably is selected from the group consisting of cocoa butter and vitebesole.

A further aspect of the present invention is the use of a blood serum of the present invention or of a pharmaceutical composition of the present invention for the production of a medicament for the treatment of a disease or condition, which can be affected by an increase of cyclic adenosine monophosphoric acid contents in the brain of the subject requiring treatment.

Another aspect of the present invention is the use of a blood serum of the present invention or of a pharmaceutical composition of the present invention for the production of a medicament for the improvement of cognitive and/or learning skills in particular improvement of the long term memory.

Another aspect of the present invention is the use of a blood serum of the present invention or of a pharmaceutical composition of the present invention for the production of a medicament for the treatment of seizures, in particular epileptic seizures.

A further aspect of the present invention is a use of a blood serum of the present invention or of a pharmaceutical composition of the present invention for the production of a medicament for the treatment of proliferative diseases and apoplexy.

In a preferred embodiment of the use of the present invention the proliferative disease is selected from the group consisting of malignomas of the gastrointestinal or colorectal tract, the liver, the pancreas, the kidney, the bladder, the thyroid, the prostate, the endometrium, the cervix, the ovary, the uterus, the testes, the skin, the oral cavity; melanoma; dysplastic oral mucosa; invasive oral cancers; small cell and non-small cell lung carcinomas; mammary tumors, in particular hormone-dependent breast cancers and hormone independent breast cancers; transitional and squamous cell cancers; neurological malignancies including neuroblastomas, gliomas, astrocytomas, osteosarcomas, meningiomas; soft tissue sarcomas; hemangioamas and endocrinological tumors, in particular pituitary adenomas, pheochromocytomas, paragangliomas, haematological malignancies, in particular lymphomas and leukemia.

And in a further preferred embodiment of the use of the present invention the proliferative disease comprises cells similar to the human T cell lymphoma cell line Jurkat, the human B cell lymphoma cell line Raji, the human melanoma cell line Bro, the human cervical cancer cell line HeLa, the human adenocarcinoma cell line MCF-7, the osteosarcoma cell line Mg63, the fibrosarcoma cell line HT1080, the neuroblastoma cell line IMR-32 and the hepatocarcinoma cell line HepG2.

In a further preferred embodiment of the use of the present invention the medicament is administered to a subject in need of treatment in an amount ranging from 50 to 150 mg/kg body weight, preferably ranging from 90 to 100 mg/kg body weight.

### DETAILED DESCRIPTION OF THE INVENTION

The present inventors have surprisingly found that the stimulation of animals, in particular chickens with electric currents and the further treatment of blood serum obtained from the blood with γ-radiation leads to a significant increase of the biological activity of the blood serum product. The resulting products are capable of positively influencing various body functions, conditions and diseases of a patient.

The beneficial effect of blood serum treated with electricshock and γ-radiation is surprising for two reasons. Firstly, it is known in the prior art that electric shocks cause severe disturbances of all vital functions and systems within the organism in particular the central nervous system, the blood and circulatory system and the respiratory system (see, for example, Orlow, A. N. et al. (1977) Medicine). Secondly, it was also discovered that blood and serum prepared from blood is sensitive to radiation and is unstable and deactivated under ionizing radiation (see, for example, Radiomedicine—M. Atomisdat (1972) 123-125 and Gergely, J. et al. (1967) Radiosterilization of Medical Products 115-124).

These studies did not lead to the results of the present invention and would have suggested that it would not be possible to obtain a biological active blood serum through the combination of electroshock and γ-radiation treatment. Thus, it was surprising that arterial and/or venous blood drawn from an animal, in particular from a chicken treated with an electroshock of grade II to grade III and with gamma radiation resulted in a biological active blood serum. The specific activities of the thus obtained blood serum will be set out in more detail below.

Accordingly, a first aspect of the present invention is a method for producing a biological active blood serum comprising the steps of:

a) electrostimulation of a non-human animal,

b) withdrawal of blood from said animal,

c) isolation of serum from said blood, and

d) gamma irradiation of said serum.

Various animals can be used in the method of the present invention, however, it is preferred that the non-human animal is selected from the group consisting of mammals and birds. Because of their easy availability it is particularly preferred to use farm animals like poultry, e.g. chicken, dug, goose, ostrich and quail. A particular preferred animal which can be used in the method of the present invention is a chicken. The type of mammal that can be used in the method of the present invention is not particularly restricted and comprises without limitation rodents, e.g. mouse, hamster and rat, cats, dogs, horses, donkeys, sheep, cows, and goats.

It is envisioned that the electrostimulation leads to the release of certain compounds within the animal which cause and/or contribute to the surprising therapeutic effect of the biological active blood serum of the present invention. The non-human animal can be stimulated in different regions of the body. Preferably the electrostimulation is carried out at the head, the neck, the body and/or on one or more of the limbs. It is possible to stimulate the body only at one position or at several positions at once. A particular preferred body part for the electrostimulation is the head of the respective animal. When stimulating birds, in particular chicken it is preferred that the head is electrostimulated.

The electrostimulation can be carried out by art known methods, preferably using metal electrodes or water baths as used, for example, during culling of cattle or electrocution of poultry. Preferably, the electrostimulation is carried out for a time period of between 1 and 60 seconds, preferably between 1 and 30 seconds, more preferably between 2 and 10 seconds, and most preferably between 3 and 4 seconds. The length of a time period will be longer in case that a large animal is electrostimulated and can be shorter in cases were small animals are electrostimulated. For example for the stimulation of chicken heads a particular preferred time period of the electrostimulation is between 2 and 10 seconds and more preferably between 3 and 4 seconds. The other variables which can be adapted during the electrostimulation of the animal is the voltage, the current and the frequency of the current and the present inventors have defined certain preferred ranges for these parameters. The actual parameter chosen will depend in part on the size of the animal as well as on the region of the animal to be stimulated. In general larger animals and larger regions will require a higher voltage and current. Thus, the electro stimulation is preferably carried out with a voltage in the range of between 50 Volt and 150 Volt, preferably 80 Volt to 120 Volt and more preferably between 110 Volt and 120 Volt. The ranges for the currents that can be applied are between 0.01 A and 0.4 A, preferably between 0.02 A and 0.1 A, more preferably between 0.04 A and 0.06 A and most preferably about 0.05 A. Voltage, current and application time are preferably chosen to administer energy in the range of between 1 and 1,000 Ws, preferably in the range of 10 to 200 Ws and even more preferably in the range of 15 to 100 Ws.

For the stimulation of the preferred animals, i.e. chicken, it is preferred that the electrostimulation is carried out with a voltage in the range of between 80 Volt to 120 Volt and more preferably between 110 Volt and 120 Volt. Furthermore, a current in the range of between 0.04 A and 0.06 A, in particular of 0.05 A is preferred in the context of the electrostimulation of birds, in particular of chicken.

In a particular preferred embodiment of the method of the present invention the electrostimulation of a bird, in particular a chicken, is carried out for between 3 and 4 seconds at a voltage of between 80 V and 120 V, in particular 110 V and 120 V. In this preferred embodiment the current is preferably between 0.04 A and 0.06 A and most preferably about 0.05 A.

The frequency of the electrostimulation does not appear to be particularly critical but is preferably in the range of between 10 and 200 Hertz, more preferably in the range of between 45 to 65 Hz and most preferably around 50 Hz.

The gamma irradiation of the serum during step d) of the method of the present invention can be carried out with any gamma source including X-ray sources and radionuclides. Preferably the gamma radiation source is a radionuclide with a defined gamma radiation pattern. Preferred sources for the gamma radiation are selected from the group consisting of ⁶⁰Co, ¹³⁷Cs, ⁶⁷Cu, ⁶⁷Ca, ¹¹¹In, ¹⁹²Ir, ^{99m}Tc and ¹⁷⁰Tm. Out of those ⁶⁰Co, ¹³⁷ Cs, ¹⁹² Ir and ¹⁷⁰Tm are particular preferred with ⁶⁰Co being the most preferred gamma radiation source, for us in the method of the present invention. The radiation dose adsorbed by the serum is in the range of between 10 to 40 kGy preferably in the range of between 15 to 35 kGy and more preferably in the range of between 20 and 30 kGy, i.e. 25±5 kGy.

The withdrawal of the blood from the animal can be effected by any art known method and includes syringes as well as puncturing of arteries or veins or decapitation in particular in the context of the withdrawal of blood from birds. It is possible to withdraw only a part of the blood or to completely with draw the blood of the animal. The later is preferably used, if a lethal dose of electricity has been applied to the animal. The withdrawn blood can be arterial and/or venous blood.

The serum can be isolated from the blood by any known method including filtration, sedimentation and centrifugation. It is, however, preferred that the blood is incubated for between 4 and 72 h at a low temperature, e.g. between 2° and 10° C., preferably between 4-8° C. to allow clotting of the blood which leads to the release of additional factors into the blood. Thus, in a preferred embodiment to the method of the present invention the method further comprises the step of incubating the blood after the withdrawal of the blood from the animal and prior to the isolation of the serum from the blood, e.g. for between 4 and 72 h at a low temperature, e.g. between 2° and 10° C., preferably between 4-8° C.

In a further preferred embodiment of the method of the present invention the method comprises the further step of lyophilization of the serum prior to the irradiation step d). The lyophilization allows easier handling of the serum during irradiation and optimizes absorption of the radiation by the serum components.

A further aspect of the present invention is the biological active blood serum itself, which is producible according to a method of the present invention. It is distinct from prior art blood serums which do not employ the steps of the method of the present invention, which is evidenced by its particular therapeutic effects, which are not exhibited by prior art blood serum products.

As it has been surprisingly found that the biological active blood serum of the present invention provides certain therapeutical effects, e.g. an anti-proliferative activity or an anti epileptic activity, a further aspect of the present invention is a pharmaceutical composition, comprising a biological active blood serum producible according to the method of the present invention. Such pharmaceutical composition can further comprise one or more pharmaceutically acceptable diluents; carriers; excipients, including fillers, binders, lubricants, glidants, disintegrants, and adsorbents; and/or preservatives.

The pharmaceutical composition of the present invention can be administered by various well known routs, including oral and parenteral administration, e.g. intravenous, intramuscular, intranasal, intradermal, subcutaneous and similar administration routes. Parenteral administration and particular intravenous administration is preferred. Depending on the route of administration different pharmaceutical formulations are required and some of those may require that protective coatings are applied to the drug formulation to prevent degradation of the biological active serum in, for example, the digestive tract.

Thus, preferably the pharmaceutical composition of the present invention is formulated as a syrup, an infusion solution, or injection solution, a tablet, a capsule, a capslet, a lozenge, liposome a suppository, a plaster, a band-aid, a retard capsule, a powder or a slow release formulation.

Particular preferred pharmaceutical forms are forms suitable for injectionable use and include sterile aqueous solutions or dispersions and sterile powders for the extemporaneous preparation of sterile injectable solutions or dispersion. In all cases the final solution or dispersion form must be sterile and fluid. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. The biological active blood serum of the present invention can also be formulated into liposomes, in particular for parenteral administration. Liposomes provide the advantage of increased half life in the circulation, if compared to the free drug and a prolonged more even release of the enclosed drug.

Sterilization of infusion or injection solutions can be accomplished by any number of art recognized techniques including but not limited to addition of preservatives like anti-bacterial or anti-fungal angents, e.g. parabene, chlorobutanol, phenol, sorbic acid or thimersal. Further, isotonic agents, such as sugars or salts, in particular sodium chloride may be incorporated in infusion or injection solutions.

Production of sterile injectable solutions containing the biological active blood serum is accomplished by incorporating the biological active serum in the required amount in the appropriate solvent with various ingredients enumerated above as required followed by sterilization. To obtain a sterile powder the above solutions are vacuum-dried or freeze-dried as necessary. Preferred diluents of the present invention are water, physiological acceptable buffers, physiological acceptable buffer salt solutions or salt solutions. Preferred carriers of the present invention are cocoa butter and vitebesole. Excipients which can be used with the various pharmaceutical forms of the biological active blood serum can be chosen from the following non-limiting list:
a) binders such as lactose, mannitol, crystaline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glycerids and sodium stearyl fumarates,
c) disintegrants such as starches, croscaramellose, sodium methyl cellulose, agar, bentonite, alginic acid, carboxymethyl cellulose, polyvinyl pyrrolidone and the like.

Other suitable excipients can be found in the Handbook of Pharmaceutical Excipients, published by the American Pharmaceutical Association, which is herein incorporated by reference.

A further aspect of the present invention is the use of a blood serum or of a pharmaceutical composition of the present invention for the production of a medicament for the treatment of a disease or condition which can be effected by an increase of cyclic adenosine monophosphoric acid content in the brain of the treated subject.

Another aspect of the present invention is the use of the biological active blood serum or of a pharmaceutical composition of the present invention for the production of a medicament for the improvement of nootropic, cognitive and/or learning skills of the treated subject and in particular the improvement of the long-term memory. The usefulness for this indication is based on the discovery that the blood serum or the pharmaceutical composition of the present invention increase the learning abilities of treated subjects.

Furthermore, the blood serum or the pharmaceutical composition can be used for the treatment of seizures of any type in particular, however, for the treatment of epileptic seizures. In particular in severe epileptic forms and during grand mal seizures the administration of the biologically active blood serum or of the pharmaceutical composition of the present invention can prevent death that is sometimes associated with severe seizures. In connection with the observation that the present invention can be used for the treatment of epileptic seizures, it has also discovered that the blood, serum or the pharmaceutical composition of this invention can be used for the treatment of nervous diseases, including without limitation, bipolar disorder, depression, anxiety related disorders, epilepsy, Alzheimer's disease, Parkinson's disease, peripheral neurophathy, cerebral amyloid angiopathy, neuro degenerative disorders and spinal cord injury.

A further aspect of the present invention is the use of the blood serum or of the pharmaceutical composition of the present invention for the production of a medicament for the treatment of proliferative diseases and apoplexy. It was particular surprising that the biological active blood serum or the pharmaceutical composition of the present invention did show a marked anti-proliferative effect, if tested on a variety of tumor cell lines in vitro. It, therefore, appears that the biological active blood serum or the pharmaceutical composition of the present invention is particular suitable for the treatment of proliferative diseases and preferred proliferative diseases which are treatable according to the use of the present invention are selected from the group consisting of malignomas of the gastrointestinal or colorectal tract, the liver, the pancreas, the kidney, the bladder, the thyroid, the prostate, the endometrium, the cervix, the ovary, the uterus, the testes, the skin, the oral cavity; melanoma; dysplastic oral mucosa; invasive oral cancers; small cell and non-small cell lung carcinomas; mammary tumors, in particular hormone-dependent breast cancers and hormone independent breast cancers; transitional and squamous cell cancers; neurological malignancies including neuroblastomas, gliomas, astrocytomas, osteosarcomas, meningiomas; soft tissue sarcomas; hemangioamas and endocrinological tumors, in particular pituitary adenomas, pheochromocytomas, paragangliomas, haematological malignancies, in particular lymphomas and leukemia.

Since the anti-proliferative effect of the biological active blood serum of the present invention or of the pharmaceutical composition of the present invention has been first established for a variety of tumor cell lines it is particular suitable for the treatment of proliferative diseases which comprise cells and/or tumor tissue comprising cells similar to the tumor cell lines used in those experiments. Accordingly, preferred prolifertive diseases treatable with the biological active blood serum or the pharmaceutical composition comprising the biological active serum comprise cells similar to the human T cell lymphoma cell line Jurkat, the human B cell lymphoma cell line Raji, the human melanoma cell line Bro, the human cervical cancer cell line HeLa, the human adenocarcinoma cell line MCF-7, the osteosarcoma cell line Mg63, the fibrosarcoma cell line HT1080, the neuroblastoma cell line IMR-32 and the hepatocarcinoma cell line HepG2. In this context the term “similar cells” are cells, which have the same origin, e.g. T-cell, B cell or neural lineage, as the respective cell line and which carry a mutation in the same or a functionally equivalent gene and wherein this mutation contributes to the proliferative activity of the cell, e.g. mutation in p53, pRb, cdc 2, cdk 4, cyclin A, cyclin B, p21^{ras}, c-fos, c-jun, p107, p130 and the like; which carry the same or a functionally similar exogenous gene, e.g. human papilloma virus (HPV), E 6 or E 7, insertion into the cyclin B promoter by hepatitis B virus and the like; or which have the same chromosomal rearrangement or abnormality, i.e. deletion, chromosomal multiplicity etc.

Based on the results in animals and in cell culture certain amounts of the biological active blood serum are preferred for the treatment of the diseases and conditions for which the blood serum and pharmaceutical composition can be employed. It is, however, understood that depending on the respective condition as well as on the respective patient to be treated, i.e. depending on the severity of the disease or condition, the general health status of the patient, etc., different doses of the biological active blood serum or the pharmaceutical composition are required to elicit a therapeutic effect. The determination of the appropriate dose lies within the discretion of the attending physician. It is contemplated that the dosage of the biologically active blood serum in the therapeutic method of the invention should be in the range of about 0.1 mg to about 200 mg serum per kg body weight. However, in a preferred use of the present invention the biologically active blood serum is administered to a subject in need thereof in an amount ranging from 50 to 150 mg/kg body weight, preferably ranging from 90 to 100 mg/kg body weight. The duration of therapy with biologically active blood serum will vary, depending on the severity of the disease being treated and the condition and idiosyncratic response of each individual patient.

The following examples are included to demonstrate preferred embodiments of the invention. It should be appreciated by those of skill in the art that the techniques disclosed in the examples that follow represent techniques discovered by the inventors to function well in the practice of the invention, and thus, can be considered preferred modes for its practise. However, those of skill in the art should, in light of the present disclosure, appreciate that many changes can be made in the specific embodiments that are disclosed without departing from the spirit and scope of the invention as set out in the appended claims. All references cited are incorporated herein by reference.

### BRIEF DESCRIPTION OF THE FIGURES AND DRAWINGS

FIG. 1: Cytotoxic effect of two pharmaceutical preparations on Jurkat cells.

The cytotoxic effect on the human T cell lymphoma cell line Jurkat is depicted for two different pharmaceutical preparations comprising biological active serum of the invention various concentrations of the. The viability of the Jurkat cells is depicted on the y-axis in percentages, while the amount of the biological active serum is indicated on the x-axis in mg/ml.

FIG. 2: Cytotoxic effect of two pharmaceutical preparations on Raji cells.

The cytotoxic effect on the human B cell lymphoma cell line Rajiis depicted for two different pharmaceutical preparations comprising biological active serum of the invention various concentrations of the. The viability of the Raji cells is depicted on the y-axis in percentages, while the amount of the biological active serum is indicated on the x-axis in mg/ml

FIG. 3: Cytotoxic effect of two pharmaceutical preparations on Bro B-19 cells.

The cytotoxic effect on the human T cell lymphoma cell line Bro B-19 is depicted for two different pharmaceutical preparations comprising biological active serum of the invention various concentrations of the. The viability of the Bro B-19 cells is depicted on the y-axis in percentages, while the amount of the biological active serum is indicated on the x-axis in mg/ml

FIG. 4: Cytotoxic effect of two pharmaceutical preparations on HeLa cells.

The cytotoxic effect on the human T cell lymphoma cell line HeLa is depicted for two different pharmaceutical preparations comprising biological active serum of the invention various concentrations of the. The viability of the HeLa cells is depicted on the y-axis in percentages, while the amount of the biological active serum is indicated on the x-axis in mg/ml

FIG. 5: Cytotoxic effect of two pharmaceutical preparations on MCF-7 cells.

The cytotoxic effect on the human T cell lymphoma cell line MCF-7 is depicted for two different pharmaceutical preparations comprising biological active serum of the invention various concentrations of the. The viability of the MCF-7 cells is depicted on the y-axis in percentages, while the amount of the biological active serum is indicated on the x-axis in mg/ml

FIG. 6: Cytotoxic effect of two pharmaceutical preparations on IMR-32 cells.

The cytotoxic effect on the human T cell lymphoma cell line IMR-32 is depicted for two different pharmaceutical preparations comprising biological active serum of the invention various concentrations of the. The viability of the IMR-32 cells is depicted on the y-axis in percentages, while the amount of the biological active serum is indicated on the x-axis in mg/ml

FIG. 7: Cytotoxic effect of two pharmaceutical preparations on HT1080 cells.

The cytotoxic effect on the human T cell lymphoma cell line HT1080 is depicted for two different pharmaceutical preparations comprising biological active serum of the invention various concentrations of the. The viability of the HT1080 cells is depicted on the y-axis in percentages, while the amount of the biological active serum is indicated on the x-axis in mg/ml

FIG. 8: Cytotoxic effect of two pharmaceutical preparations on HepG2 cells.

The cytotoxic effect on the human T cell lymphoma cell line HepG2 is depicted for two different pharmaceutical preparations comprising biological active serum of the invention various concentrations of the. The viability of the HepG2 cells is depicted on the y-axis in percentages, while the amount of the biological active serum is indicated on the x-axis in mg/ml

FIG. 9: “Bell” curve of the proliferative activity of cells treated with mitogens

The curve of the lymphocytes is assayed on the basis of the amount of DNA biosynthesis in relation to the concentration of the mitogen proliferative activity of. The DNA biosynthesis activity is measured by the amount of incorporated radioactivity and the acid insoluble counts per minute.

FIG. 10: Mitogenic activity of the pharmacological composition.

Depicted is the mitogenic effect of the pharmaceutical composition on the amount of DNA biosynthesis within a substance concentration range from 0.1 to 100.0 mg/ml. The amount of DNA synthesis is assessed on the basis of the amount of radioactivity incorporated into the DNA.

FIG. 11: Effect of pharmacological composition on MCF-7 cells.

Depicted is the amount of DNA synthesis in the human milk gland carcinoma cell line MCF-7 in relation to the amount of biological active serum comprised in the pharmacological composition.

### EXAMPLES

### Example 1

Method for Obtaining of Chicken Blood Treated by Electroshock

For the preparation of serum from chicken, the chicken were treated with an electroshock of grade II to III (electrical voltage 80-120 V, current 0.05 A, frequency 50 Hz, application time: 3 to 4 sec at the head). Blood was then drawn from the arteria carotis and further incubated at a temperature of 4-8° C. for 18-24 h in polyethylene flasks. After complete retraction of blood clots the flasks were spun at 3.000 rpm for between 20-30 minutes. The serum was separated from the blood clots and lyophilized under art known conditions. The flasks with the lyophilized serum were treated on a RZ-100-M apparatus with 20-30 kGy, preferably at around 25 kGy using ⁶⁰CO as a gamma radiation source. The treated serum was stored at a temperature of between 4-8° C. for later use.

Evidence for the Stimulating Effect of Chicken Blood Serum Treated with Electroshock

The below experiments were carried out using male Wistar rats with an average body mass of 280-300 g. The animals were randomly assigned to 4 groups of 10 rats each. The first group was administered with 1.0 ml of a physiological salt solution. The second group was administered a dose of 100±5.0 mg/kg body weight of the blood serum of the present invention within 1.0 ml solution. 30 minutes after the injection the rats were decapitated. 1 ml of a physiological salt solution was administered to the rats of the third group and the rats of the fourth group received biological active serum (100±5 mg/kg body weight) in an amount of 1.0 ml solution. 30 minutes after the injection the animals, which had a weight attached to their tail (10% of the body weight of the rat), were placed in a basin with water (25° C.). After the first signs of agony the animals were removed from the water and decapitated.

The brain, heart, liver (all being organs, which are exposed to an extensive energetic strain in processes of extreme adaptation) as well as the skeletal muscles (as mainly effected organ) were taken as samples for further analysis. The tissue samples of each rat were weight, cooled with an isotonic NaCl solution and rapidly frozen in liquid nitrogen. The complete time which elapsed between the application “stress” and the final processing of the samples was in the range of between 5 to 6 minutes maximally.

The amount of adenosine triphosphoric acid, adenosine diphosphoric acid and adenosine monophosphoric acid in the skeletal muscles of the rats was determined. Nucleotides were separated by means of ionexchange chromatography on columns employing Anionit Dowex 1. The determination of the amount of adenosine triphosphoric acid, adenosine diphosphoric acid and adenosine monophosphoric acid was carried out spectophotometrically at a 256 nm range (spectrophotometer Hitachi-557). The energy potential was determined according to the following formula
(ATP−0.5ADF)/(ATP+ADP+AMP).

The determination of the amount of cyclic adenosine monophosphoric acid in the brain, heart and liver was carried out using known radioimmunology analysis with a detection apparatus of Amersham (Great Britain).

The determination of the labelled adenosine monophosphoric acid was carried out using art known methods employing the scintillation counter GS-8.

Results

The various amounts of adenosine triphosphoric acid, adenosine diphosphoric acid and adenosine monophosphoric acid and the increase of the energy potential are apparent both when comparing the third group with either the control group one or the second group (p<0.05) as well as when comparing the fourth group with either the control group one or the second group (p<0.05) and further when comparing the third and the fourth group among each other (p<0.05) (see also Table 1).

TABLE 1
Amount of adenosine triphosphoric acid, adenosine diphosphoric acid and adenosine
monophosphoric acid and the energy potential within the tissue of the skeletal
muscles with and without the administration of the biological active serum

| | | | | Energy |
|---|---|---|---|---|
| | Amount of nucleotides | | | potential |
| | (μmol/l/g tissue) | | | (conditional |
| Group of rats | ATPh M ± m | ADPh M ± m | AMPh M ± m | coefficient) |
| I (physiological | 7.55 ± 0.19 | 0.95 ± 0.12 | 0.25 ± 0.08 | 0.917 ± 0.080 |
| salt solution − | (7.28-7.85) | (0.79-1.18) | (0.14-0.37) | |
| control) n = 10 | | | | |
| II (serum) | 7.89 ± 0.12 | 1.12 + 0.11 | 0.14 ± 0.05 | 0.923 ± 0.125 |
| n = 10 | (7.65-8.02) | (0.93-1.27) | (0.08-0.25) | |
| III (physiological | 1.34 ± 0.08 | 3.56 ± 0.17 | 0.78 ± 0.07 | 0.549 ± 0.014 |
| salt solution + | (1.19-1.49) | (3.21-3.81) | (0.65-0.93) | |
| swimming) n = 10 | | | | |
| IV (serum + | 4.78 ± 0.17 | 2.55 ± 0.11 | 0.33 ± 0.09 | 0.790 ± 0.095 |
| swimming) n = 10 | (4.35-4.95) | (2.36-2.72) | (0.19-0.48) | |

The amounts of adenosine phosphoric acid determined during the active period and 30 minutes after mild stress because of the injection (control group I) confirm that under the influence of serum a mixing of the amount of adenosine triphosphoric acid and adenosine diphosphoric acid occurs at the upper norm values in the muscle tissue, while at the same time the amount of adenosine monosphoric acid decreases.

These results can be interpreted in such that the biological active serum drives the increase of the energetic potential within the muscle tissue. The calculation of the energy potential confirms this tendency.

Under the influence of an extreme swimming stress in the third group (physiological salt solution+swimming) a decrease of adenosine triphosphoric acid and an increase of the content of adenosine diphosphoric and adenosine monophosphoric acid in relation to the control group was determined.

Under extreme stress the rats of the fourth group (serum+swimming) the general tendencies of the changes of the amounts of adenosine triphosphoric, diphosphoric and monophosphoric acid observed in the third group remained similar, however, the amount of adenosine triphosphoric acid stayed significantly higher and when calculating the energy potential an increase in the energy potential of 43% (p<0.05) in comparison to the third group was observed.

A comparative analysis of the cyclic adenosine monophosphoric acid in heart and liver tissue as well as in brain tissue of rats of the first and second group demonstrated that under the influence of serum an increase of the amount of cyclic monophosphoric acid can be detected in the brain tissue (p<0.01) while in heart and liver tissue no significant difference in comparison to the control group was determined (see Table 2).

TABLE 2
Amount of cyclic adenosine monophosphoric acid in brain, heart and
liver tissue after the administration of biological active serum.

| | Amount of cyclic adenosine monophosphoric acid | | |
|---|---|---|---|
| | (pmol/l/g raw tissue) | | |
| Group of rats | brain M ± m | heart M ± m | liver M ± m |
| I (physiological | 3.95 ± 0.58 | 3.07 ± 0.11 | 2.75 ± 0.18 |
| solution − con- | (3.32-4.55) | (2.98-3.32) | (2.47-2.99) |
| trol) n = 10 | | | |
| II (serum) n = 10 | 4.58 ± 0.23 | 3.26 + 0.16 | 2.98 ± 0.99 |
| | (4.26-4.85) | (3.02-3.47) | (2.95-3.12) |
| III (physiologi- | 0.82 ± 0.13 | 0.49 ± 0.17 | 1.01 ± 0.07 |
| cal solution + | (0.66-1.05) | (0.32-0.78) | (0.86-1.14) |
| swimming) | | | |
| n = 10 | | | |
| IV (serum + | 1.58 ± 0.13 | 0.83 ± 0.14 | 1.27 ± 0.08 |
| swimming) | (1.38-1.76) | (0.56-0.96) | (1.12-1.41) |
| n = 10 | | | |

Under the influence of extreme stress a significant reduction (p<0.05) of the amount of cyclic adenosine monophosphoric acid was observed in all tissues of rats of the third group (physiological salt solution+swimming) as well as of the fourth group (serum+swimming). This decrease was, however, lessened under the influence of biological active serum. Accordingly, the amount of adenosine monophosphoric acid in brain tissue was 92% higher as the respective amount of the third group (p<0.05), 96% higher in heart tissue and 25.7% higher in liver tissue.

Consequently, the serum of the present invention treated with electroshock and γ-radiation increases the energy potential in skeletal muscles of rats, increases the amount of cyclic adenosine monophosphoric acid in brain tissue both during rest phases as well as under extreme physical stress and facilitates the increase of cyclic adenosine monophosphoric acid in heart and liver tissue after physical stress of rats.

### Example 2

Long-Term Memory.

In this experiment 150 male Wistar rats were used. All rats were previously tested with a classical “open field” test and were assigned according to the activity into three groups: active, medium activity and passive.

For the establishment of a situation reflex rats were placed in a cylinder which was located within a thermostatically controlled basin. The cylinder was placed on three supports and allowed the animals to dive under the lower edge of the cylinder to reach a platform located outside of the cylinder.

The latent time for the first experiment as well as the latent time of the final solving of the task of “escaping” from a closed room and from the water was determinable.

When establishing the situation reflex the rats were divided into three groups:
“fast diver”: those rats, which were able to find the exit within the first minute
“slow divers”: those rats which only started to look for the exit after 2 to 3 minutes
rats, which were not willing to solve the problem within 10 minutes

The biological active serum was injected into the peritoneal cavity 30 minutes prior to the onset of the experiment in a dose of 100 mg/kg body weight. 1 ml of a physiological salt solution was administered to the control animals. On the next day the situation reflex behaviour was tested and afterwards the rats were given a 40 day pause.

According to this method 120 rats—60 control rats and 60 rats receiving biological active serum—were “educated”. The rats were previously assigned to three groups as another above, i.e. active, medium activity and passive (20 animals within each group) furthermore they were subdivided for this analysis in “fast” and “slow” divers.

Results

Some rats did not show a “diving reflex” at all (Table 3). Within the control group six of the active rats and rats of medium activity and 12 animals of the passive group did not show the “diving reflex” at all. On the second day this number remained the same for the active rats and for rats of medium activity, however, declined for the passive animals to 7. After 42 days the same 11 rats of the passive group refused to dive under the edge of the cylinder (1 rat died during the experiment).

For the active rats of the rats receiving biological active serum there was not a single rat that declined to solve the problem on the second day. For all the rats of medium activity three refused to solve the problem on the first day but nevertheless solved it at the second day and continued to have this capability even after 42 days. Among the passive rats of the rats receiving serum 4 rats were not willing to solve the problem, however, at the second day this amount was reduced to 2 and remained constant even after 42 days.

TABLE 3
Number of rats which did not show a “diving reflex”

1 day (24 hours)

| Test animal | | 1 | 2 | 42 |
|---|---|---|---|---|
| Active rats | Control | 6 | 6 | 6 |
| and rats of | Serum | 3 | 0 | 0 |
| medium activity | | | | |
| Passive rats | Control | 12 | 7 | 11* |
| | Serum | 4 | 2 | 2 |

*one rat died during the experiment

Within the group of the “fast divers” a single serum injection within the 40 day time period of the experiment did not show a significant change in maintaining the situation reflex.

For the “slow divers” there was a statistically significant capability to maintain the ability to quickly find an escape route from an extreme situation (Table 4). Thus, the time of reflex formation on the second day decreased in comparison to the first day for both the control group and the trial group. In the control group these reflexes were completely gone after 42 days while for animals which had received an injection of biological active serum the reflex completely remained. In addition there was a difference in the time required for the reflex, which was 2-3-fold higher (p<0.01) for control rats.

TABLE 4
Influence of blood serum on the formation and maintenance of the situation reflex in “slow divers” (in sec.)

| | 1 day (24 hours) | | | | | | |
|---|---|---|---|---|---|---|---|
| | 1 | | | | | | |
| Test | | LP-1 | | 2 | | 42 | |
| animal | | (latency period) | LP-2 | LP-1 | LP-2 | LP-1 | LP-2 |
| Active | Control | 125.7 + 25 | 176.7 + 19.6 | 45.5 + 9.9 | 59.0 + 8.8 | 37.5 + 30.0 | 181.5 + 15.5 |
| group | Serum | 111.3 + 39.7 | 164.8 + 33.5 | 55.3 + 15.6 | 68.4 + 18.8 | 3.8 + 29.3 | 55.7 + 37.28 |
| Group of | Control | 144.8 + 21.8 | 191.2 + 16.3 | 49.6 + 11.2 | 62 + 14.3 | 3.8 + 24.0 | 19.2 + 9.8 |
| medium | Serum | 118.2 + 71.6 | 178.6 + 76.8 | 3.8 + 50.3 | 77 + 54.1 | 9.2 + 28.8 | 54.6 + 39.7 |
| activity | | | | | | | |
| Passive | Control | 53.3 + 25.7 | 189.6 + 12.6 | 53 + 15.0 | 66.6 + 8.0 | 149.3 + 24.7 | 196.0 + 11.1 |
| group | serum | 18.2 + 71.6 | 178.6 + 76.8 | 63.8 + 50.3 | 77 + 54.1 | 39.2 + 28.8 | 54.6 + 39.7 |

During the formation and the long-term maintenance of information a significant role is attributed to H-cholino-ceptive mechanisms. 30 animals who were taught for five days how to dive under a “bell” were subdivided into three groups.

A physiological solution of zytisin (a H-cholino-ceptive antagonist of the brain) was injected with a dose of 1 mg/kg body weight into the peritoneal cavity 30 minutes prior to the test of the animals of the first group (ten rats).

Biologically active serum was administered in a dose of 100 mg/kg body weight to the animals of the second group (ten rats) and 30 minutes later a zytisin solution was administered with a dose of 1 mg/kg body weight.

The third animal group (control—ten rats) received a 1 ml injection of a physiological salt solution.

The complex behaviour of the animals was tested according to above-indicated method 48 hours after the injection of the indicated substances. The biotests showed 48 hours after the application of zytisin a marked slowing of the “escape reaction” from the closed room namely a 3-fold slowing in comparison to control animals.

The administration of the serum prior to the zytisin injection not only alleviated the effect of this antagonist but also led to an increase in the “escape” reaction by 20% in comparison to the control group and the overall effect of the biological active serum exceeded the effect of the antagonist by 5-fold.

TABLE 5
Latency period for the reaction of rats to the situation
in connection with the administration of zytisin
and biologically active serum (sec.)

| | | Administration | Administration |
|---|---|---|---|
| Time | Control | of zytisin | of serum + zytisin |
| parameter | (third group) | (first group) | (second group) |
| After | 13.33 ± 0.58 | 40.0 ± 14 | 8.3 ± 5.5 |
| 48 h | | <0.05 | >0.05 |
| | | | <0.05 |

On the basis of the results it was determined that brain H-cholino receptors play a role in the restoration of the “escape reaction” in connection with the modelling of a complex behaviour of animals and that the biological active serum prevents the establishment of attenuated learning.

The biologically active blood serum, therefore, stimulates the formation of long-term memory and exhibits this effect in particular in animals with a slowed down escape reaction from a closed room and out of water. Furthermore, it appears that the H-cholino-ceptive brain mechanisms play an important role in the maintenance of long-term memory and that the effects of substances negatively effecting H-cholino-ceptive brain mechanisms can be antagonized by the biologically active serum of the present invention.

### Example 3

Epilepsy

It is known in the state of the art that camphor at a toxic dosage leads to hyperactivation of the motoric area of the central nervous system which in turn causes the development of tonic cramps. Because of this camphor is used besides corazole in animal models for for the generation of cramps. Dependent on the doses of the administered camphor it is possible to cause all aspects of a small and large epileptic seizure including grand mal seizures.

In the experiments the influence of biologically active serum on the epileptic activity, which was induced by injecting camphor into the peritoneal cavity of rats was investigated. 40 male Wistar rats with a weight of 190-210 g were used in this experiment. A solution of camphor oil (20%) was injected into the peritoneal cavity in an amount of 0.25 and 0.5 ml (using 20 rats). The serum was injected at a dosage of 100±5.0 mg/kg body weight (using 20 rats).

The seizures were observed and assessed by experts. The following parameters were assessed. The latency time of the reaction, the type of cramp reaction (tonic-klonic, large and small seizures), the length of the epileptic seizure and the time intervals between them, the loss of normal mobility and the final result (death of the animal or the recovery from the pathological condition). The results are summarized in Table 6.

TABLE 6
Effect of biologically active serum
on experimentally induced epilepsy

| | | | Test group |
|---|---|---|---|
| | | | (camphor + |
| | | | serum in a |
| Doses of | Epileptic | Control group | dosage of 100 |
| camphor | seizures | (camphor) | mg/kg) |
| 0.25 | Occurrence of tonic | 7′20″ ± 10″ | 5′30″ ± 15″ |
| | cramps | | |
| | Occurrence of klonic | 12′30″ ± 32″ | 8′45″″ ± 28″ |
| | cramps in the form | | |
| | of grand mal | | |
| | seizures after | | |
| | Medium amount of | On in 6′-7′ | One in 8′-9′ |
| | seizures | | |
| | Length of seizures | 70′-80′ | 15″-20″ |
| | Condition of the | All survived | All survived |
| | animal after 48 | | |
| | hours | | |
| 0.5 ml | Occurrence of tonic | 5′40″ ± 30″ | 5′00″-32″ |
| | cramps | | |
| | Occurrence of klonic | 8′30″ ± 27″ | 8′40″ |
| | cramps in the form | | |
| | of grand mal | | |
| | seizures after | | |
| | Medium amount of | One in 5′ | One in 7′ |
| | seizures | | |
| | Length of seizures | 90″-120″ | 25″-30″ |
| | Condition of the | 60% died | All survived |
| | animal after 48 | | |
| | hours | | |

The biological active serum at a dosage of 100 mg/kg was administered in the latency period of epileptic seizures caused by injection of camphor decreased all signs of the seizure activity: the latency time of the seizure activity was increased, the klonic cramps are less severe and without loss of normal mobility and additionally the serum treated with electroshock prevented animals in the model of severe epilepsies from death (all animals survived the administration of 0.5 ml of a 20% camphor solution).

### Example 4

Effect of the Blood Serum of the Present Invention on Human Cell Proliferation.

The blood serum of the present invention is a lyophilized chicken blood treated with electroshock of grade II to III and γ-radiation. The biological active serum was used in two formulations types: (i) serum resuspended in water at a concentration of 100 mg/ml (trial fraction 1) and (ii) the supernatant of the suspension of 100 mg/ml biological active serum in water after three minute of centrifugation of the suspension at 10.000×g (trial fraction 2).

Cell Culture

The cells of the Jurkat and Raji cell line as well as lymphocytes of the human peripheral blood were cultured in plastic tissue culture plates (Nunc or Falcon) in 1640-RPMI medium (Sigma) containing 10% fetal calf serum (FCS, Gibco), 100 units/ml penicilin and 100 μg/ml streptomycin at a temperature of 37° C., at 5% CO₂ content and 95% humidity. The cell lines Bro, HeLa, MCF-7, Mg63, HT1080, IMR-32 as well as HepG2 were cultivated as above, however, using DMEM medium (Sigma) instead of 1640-RPMI medium.

Isolating of Mononuclear Leucocytes (ML) According to the Method of Boyum

Mononuclear leucocytes were isolated according to the method described by Boyum A. (Isolation of mononuclear cells and granulocytes from human blood, 1968, Cand. J. Lab. Clin. Invest., 120 (97): 9-18).

15 ml Ficoll-pack-solution was placed in each of two conical test tubes (Falcon) and subsequently 25 ml of blood two fold diluted in phosphate-bufferd saline (PBS) was applied to the Ficoll. Then the test tubes were spun at 400×g and 20° C. for 30 minutes in a Baket-Rotor. The upper phase comprising plasma was not used.

The mononuclear leucocytes (ML) which concentrated at the interface between plasma and separation medium were carefully sucked off with a pipette and collected in a centrifuge tube. Thereafter the cells were washed twice with PBS by spinning the cells at 250×g for 10 minutes and eventually suspending them in culture medium. This fraction comprised between 10-30% monocytes and 80-90% lymphocytes which are in the following termed “lymphocytes”. One part of the lymphocytes was used for studying the mitogenic activity of the biological active serum of the present invention and another to study proliferation. For this purpose phytohemaglutinine was added at a concentration of 20 mg/ml to cell suspension.

Evaluation of the Amount of Desoxyribonucleic Acid Synthesis (DNA) in Cells

To evaluate the synthesis of DNA ³H-thymidin was added to the cells and the acid insoluble fraction was evaluated for incorporated counts. Briefly, lymphocytes were incubated in 96 well plates with 200 μl medium comprising between 200 to 800×10³ cells, To each well different concentration of the biological active serum of the present invention was added. ³H-thymidin (1 μCi/Well, 40 mCi/mmol/l) was added two hours prior to termination of the incubation. For the radiometric assay the cells were harvested on filters with an automatic device for cell harvesting. The acid soluble products were washed away with 5% trifluor acetic acid (H₂O) and the radioactivity of the substances retained was measured with a scintimeter. The amount of DNA biosynthesis was measured in counts/min.

Determination of Cell Survival After Incubation with Different Substances Using the MTT-Test

The MTT-test was carried out as described by Mosmann T. (Rapid colorimetric assay for cellular growth and survival: application to proliferation and cytotoxicity assays (1983) J. Immunol. Meth. 65: 55-63).

To carry out the test cells were collected in the log. phase (adherent cells were collected when they filled about half of the tissue culture plate). They were placed into growth media in a Gorjaew-chamber, counted and subsequently resuspended in medium at a concentration of 50-100×10⁶/ml. The cell solutions were placed into the 96 well plates after the addition of the various concentrations of the trial fractions in a total amount of 100 μl. For counting of viable cells 50 μl of a solution of 3-4,5-dimethyl thiazole-2-yl)-2,5 diphenyl tetrazolium bromide (MTT) in culture medium was added to each of the different 96 well plates after termination of incubation. For the preparation of the MTT solution 1 ml MTT stock solution was mixed with 4 ml culture medium. The preparation of the MTT stock solution was carried out by the dissolving MTT in PBS (PBS comprises 0.01 mmol/l sodium phosphate buffer, pH 7.4, with 0.15 mmol/l NaCl) at a concentration of 5 mg MTT/ml followed by filtration through a filter with a pore size of 0.45 μm. The stock solution was stored at +4° C. for up to one month. After addition of the MTT solution the tissue culture plates were further incubated for 4 hours in the incubator under identical conditions. As a next step culture medium was removed by suction with a pump and 150 μl dimethyl sulfoxide (DMSO) were added to each well to dissolve blue formazan crystals, which had formed, and the optical density of the solution in each well was detected with a multichannel spectrophotometer with a microplate reader at a wave length of 540 nm (Labsystem). The viability of the cells in relation to the concentration of the added trial fraction 1 and 2, respectively, is indicated in percentages of survival of control cells. The data was processed with the computer software “Origin”.

Results and Discussion

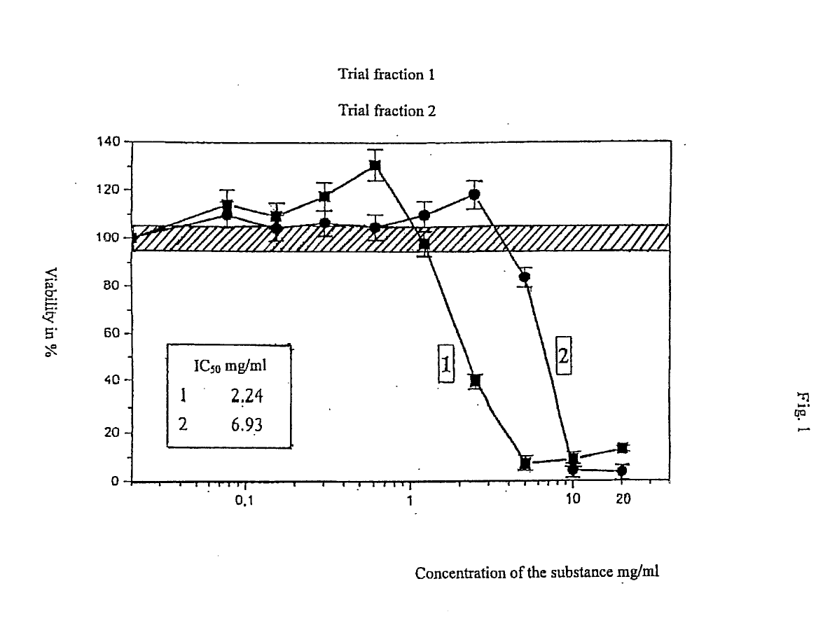

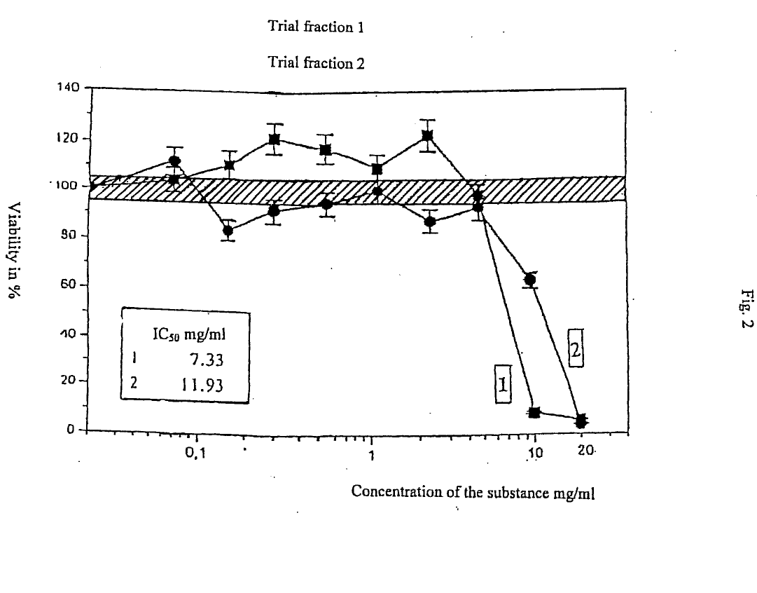

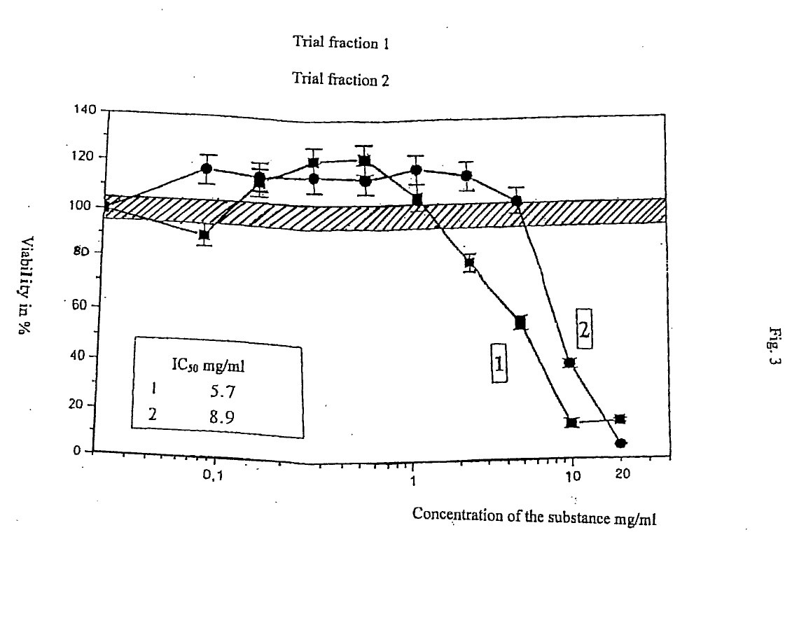

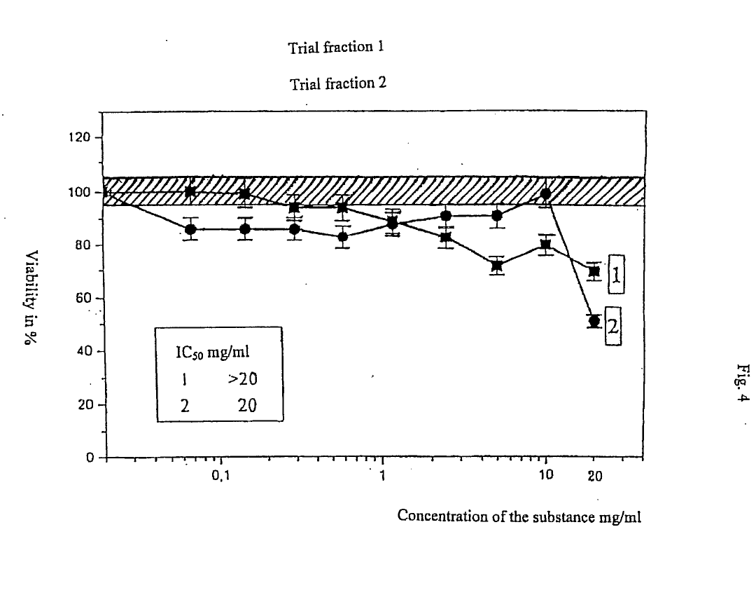

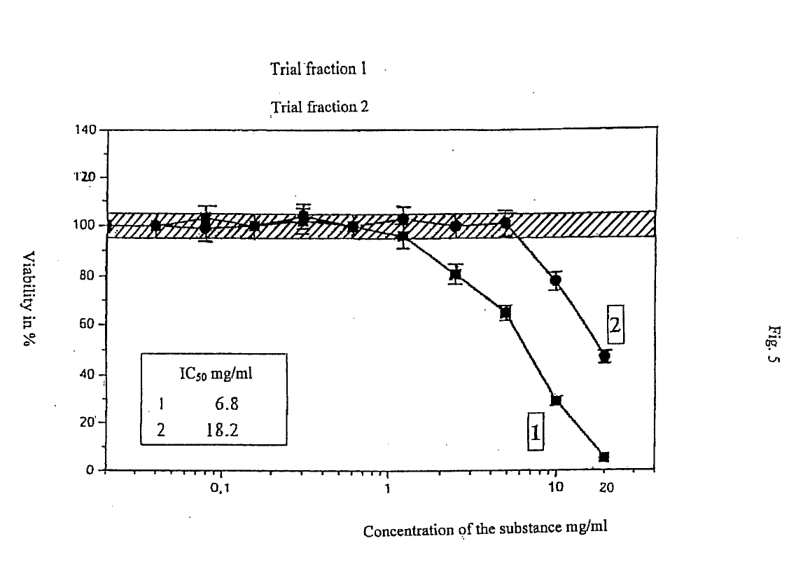

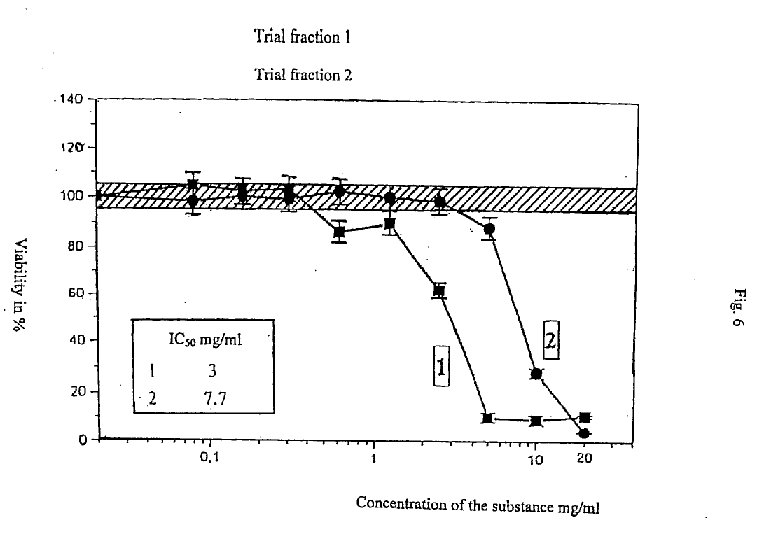

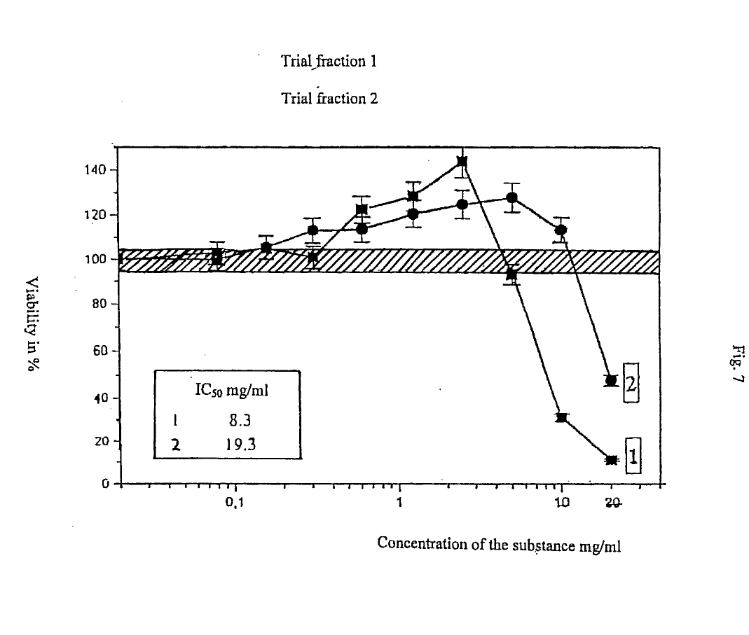

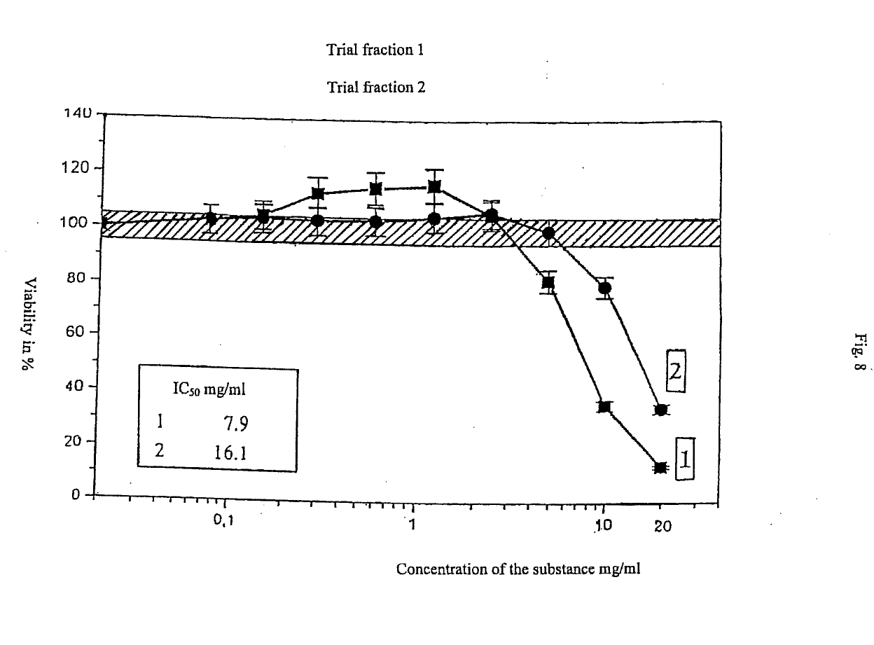

The results are depicted on FIGS. 1-8. From these results the following can be derived: a high concentration of the biological active serum of the present invention (2.5-20 mg/ml) had an inhibiting effect on all cell lines, however, the reaction of cells from different tissue types to trial fraction 1 and 2, respectively, is different. The IC₅₀ dose (concentration of the substance which leads to 50% inhibition of the cells) is substantially different among the cell lines ranging from 2.2 (Jurkat) to >20 mg/ml (Mg63) for trial substance 1 and from 3.6 (lymphocytes of the peripheral blood) to >20 mg/ml (Mg63 and HeLa) for the soluble fraction of the blood serum of the present invention (trial fraction 2). For all cell lines the toxicity of the starting substance (trial fraction 1) was above the toxicity of the soluble substance (trial fraction 2). However, it should be mentioned that the toxicity observed for the individual cell lines was almost identical for both trial fractions (Mg63, Rajii, lymphocytes of the peripheral blood) and also for the others (Jurkat, MCG-7, IMR-32) again there were 2-3-fold differences in the IC₅₀. The sensitivity of the cells to the trial fractions in comparison to the common cancer agent doxorubicin is depicted in Table 7.

TABLE 7
Sensitivity of the cell lines tested against doxorubicin

| Cell line | IC₅₀, mmol/ml |
|---|---|
| Jurkat | 100 |
| Raji | 20 |
| Bro B-19 | —** |
| HeLa | 400 |
| MCF-7 | 150 |
| Mg63 | —** |
| HT1080 | —** |
| IMR-32 | 4 |
| HepG2 | 100 |
| Lymphocytes of the peripheral blood | 40 |

Comments:
*— mmol/l = 0.58 mg/ml
**— the sensitivity against doxorubicin was not tested.

In addition it was observed that in a concentration range from 0.3 to 3 mg/ml depending on the cell line and the form of the substance (i.e. trial fraction 1 or trial fraction 2) the agent had a stimulating effect on some cells. A stimulating effect of the serum of the present invention was observed for Jurkat, Raji, Bro B-19, Mg63, HT1080 and HepG2 cells. The stimulating effect was insignificant, (10, 20, 40%) but present for both forms of the test substance. No stimulation was observed for cells of the cell line HeLa, MCF-7, JMR-32 and for lymphocytes of the peripheral blood. The stimulating effect of the test substance in its first form was observable at a much lower concentration as with the soluble fraction (trial fraction 2). Possibly the reason for this stimulation is not the stimulation of proliferation as such but rather an increase of the respiration of the cells which would also be detectable with the MTT method used for evaluating the viability of cells. The question of whether the observed stimulatory effect was due to a stimulation of proliferation or due to an increase in respiration required some further study.

When studying the interaction between different substances with immune competent cells in general at least two questions have to be discussed:
1. Does a test substance have a mitogenic activity, i.e. does it have an ability to stimulate lymphocyte proliferation (in this case the increase of the amount of the substance usually leads to an increase of the DNA biosynthesis of the lymphocytes which can be evaluated by the incorporation of ³H-thymidin)?
2. Does a test substance have a toxic effect (this question is usually determined by the inhibition of lymphocyte proliferation as assayed by the amount of incorporated ³H-thymidin or using vital dyes of the MTT-type on lymphocytes which were previously stimulated with mitogens)?

Furthermore, it has to be mentioned that non activated lymphocytes do not proliferate in culture and that the amount of proliferation only increases with increasing amounts of mitogens added to the culture medium. This is reflected in the increase in radioactivly labelled DNA.

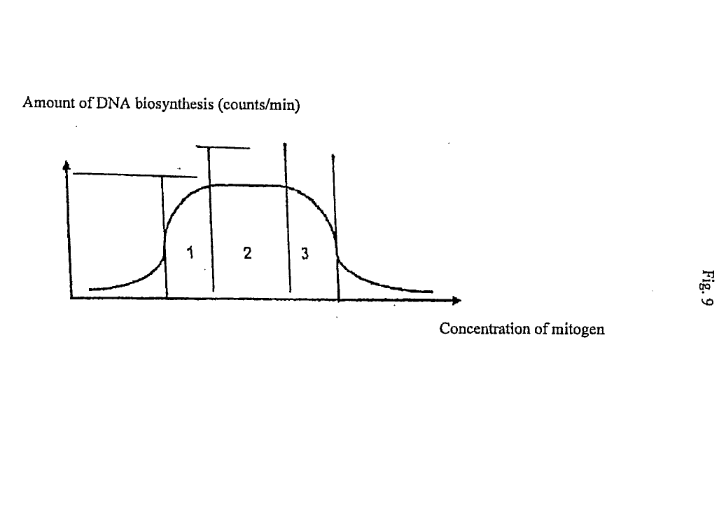

The effect of mitogens on lymphocytes in relation to the administered doses cam be depicted by a so called “bell curve” (see FIG. 9). The first section of the bell curve reflects the range of the mitogen concentration wherein an increase of mitogen leads to an increase of proliferation (as measured by DNA biosynthesis) and wherein, therefore, direct relation between mitogen concentration and proliferation exists. The second section of the curve (2) shows a saturation effect wherein a further increase of the mitogen concentration does not lead to a further increase of proliferation, i.e. the mitogen has already elicited its maximal effect. A cytotoxic activity is not yet observed. Section (3) shows the range of the mitogen concentration wherein the mitogen exerts an increasingly cytotoxic effect on the lymphocytes.

The assessment of the mitogenic activity of the trial fractions 1 and 2 was carried out in two experiments:
1. The subject matter of this experiment was the determination of the effect of the trial fractions on non-activated lymphocytes of human peripheral blood. To this end the correlation of the proliferative activity (i.e. the effect) with the administered doses was determined.
2. The subject matter of this experiment was the determination of the effect of the trial substance on the amount of DNA synthesis of lymphocytes activated by phytohemaglotinin (FHA 20 μg/ml) in a second phase.

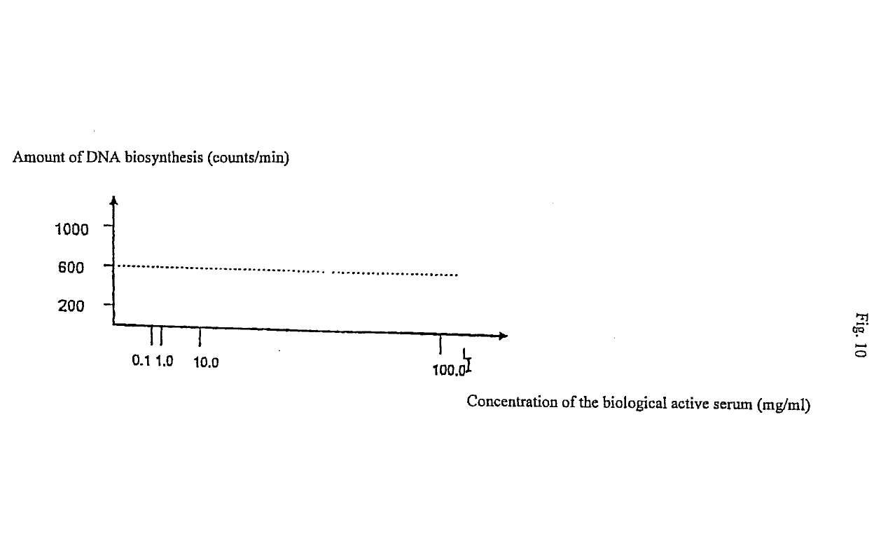

When examining the substance no mitogenic activity was observed, i.e. within a substance concentration range of 0.1-100 mg/ml no stimulation of DNA biosynthesis of lymphocytes of the peripheral blood was observed (FIG. 10).

Upon increase of the dose of the trial fractions the number of lymphocytes of the peripheral blood did not change significantly. When assessing the effect of the trial fractions on lymphocytes, which had been subsequently activated with 20 mg/ml FHA, an inhibition of activated lymphocytes by the trial fractions was observed.

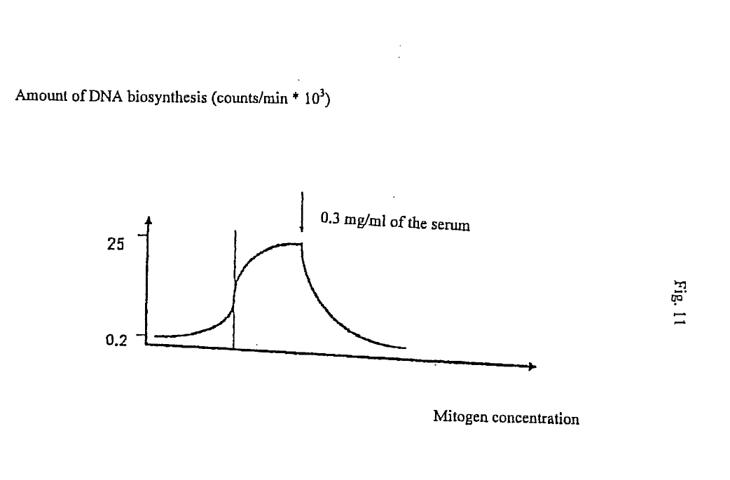

Within the experiments it was determined that trial fractions inhibited DNA biosynthesis of the stimulated lymphocytes at a concentration of 0.3 mg/ml (FIG. 11). However, the mechanism of action still needs to be determined. Further experiments to elucidate the cytostatic and the cytotoxic effect are therefore important.

To determine how the results obtained in cell culture relate to the treatment of human subjects it is important to determine the substance concentration in different organs or tissues in animal experiments. In this context it is important to investigate which different concentrations of the biologically active serum of the present invention is capable to influence DNA synthesis in organs that are involved in the lymphogenesis. Thus, it is important to determine the concentration of the biological active serum in particular in bone marrow, spleen and thymus of the mouse.

Apart from lymphocytes the cytotoxic activity of the serum of the present invention was also investigated using cells of the human milk gland carcinoma cell line MCF-7 that were spread into a monocellular layer. Because of contact inhibition no proliferation was observed for the cells. In such a model the toxic effect of a substance can be determined, i.e. it is possible to differentiate the cytotoxic effect from the cytostatic effect. When using this model it was determined that the serum of the present invention had a cytotoxic effect in a concentration of 2.5 mg/ml independent of the fact of whether the insoluble fraction of the serum of the present invention was removed or not, i.e. independent of whether trial fraction 1 or 2 was used.

It is notable that the toxic effect is markedly decreased when incubating the serum of the present invention with proliferating cells which potentially is connected with the fact that the effect was measured 24 hours after administration and not 72 hours after administration as was the case for proliferating cells (a prolonged incubation of monolayer cells can lead to death of control cells).

The above results show that the biologically active serum of the present invention has both cytostatic effects and a cytotoxic activity. The cytotoxic activity was, however, less pronounced as the combined cytotoxic and cytostatic activity albeit it was observed at the same concentration.

Taken together, blood serum from animals treated with electroshock of grade II-III and the subsequently prepared biological active substance leads to a 10-40% increase proliferation of the human cell lines Jurkat, Raji, Bro B-19, Mg63, HT1080 and HepG2, if compared to a control group. In HeLa, MCF-7, JMR-32 and lymphocytes of the peripheral blood the biologically active serum of the present invention exhibited a significant inhibition of proliferation of all tested human cancer cell lines at higher dosages (2.5-20 mg/ml). The sensitivity of the cells towards the biological active serum of the present invention is much stronger than the sensitivity against the widely used anti-cancer agent doxorubicin. Furthermore, when using the DNA biosynthesis as assessed by ³H-thymidin incorporation as a marker the substance of the present invention has both a cytostatic and a cytotoxic effect.

1. A method for producing a biologically active blood serum comprising:
a) electrostimulating a non-human animal
b) withdrawing blood from said animal,
c) isolating serum from said blood, and
d) gamma irradiating said serum.

2. The method according to claim 1, wherein the non-human animal is selected from the group consisting of mammals and birds.

3. The method according to claim 2, wherein the bird is selected from the group consisting of chicken, duck, goose, ostrich, and quail.

4. The method according to claim 1, wherein the head, the neck, the body, one or more limbs, or a combination thereof, of the non-human animal is (are) electrostimulated.

5. The method according to claim 1, wherein a) is carried out for a time period of between 1 and 60 seconds.

6. The method according to claim 1, wherein the non-human animal is electrostimulated with a voltage in the range of between 50 V and 150 V.

7. The method according to claim 1, wherein the non-human animal is electrostimulated with a current in the range of between 0.01 A and 0.4 A.

8. The method according to claim 1, wherein the non-human animal is electrostimulated with a frequency in the range of between 10 and 200 Hz.

9. The method according to claim 1, wherein said gamma irradiation provides an absorbed radiation dose of between 15 to 35 Gy.

10. The method according to claim 1, wherein the source of the gamma radiation is selected from the group consisting of ⁶⁰Co, ¹³⁷Cs, 67Cu, 67Ga, ¹¹¹In, 192Ir, ^{99m}Tc and ¹⁷⁰Tm.

11. The method according to claim 1, wherein the method further comprises incubating said blood prior to c).

12. The method according to claim 1, wherein the method further comprises lyophilization of said serum prior to d).

13. The method according to claim 1, wherein said blood is arterial blood venous blood, or a combination thereof.

14. A blood serum produced according to the method of claim 1.

15. A pharmaceutical composition comprising a blood serum according to claim 14 and one or more pharmaceutically acceptable diluents; carriers; excipients; preservatives; or a combination thereof.

16. The pharmaceutical composition according to claim 15, wherein the composition is formulated as a syrup, an infusion or injection solution, a tablet, a capsule, a caplet, a lozenge, a liposome, a suppository, a plaster, a band-aid, a retard capsule, a powder, or a slow release formulation.

17. The pharmaceutical composition according to claim 15, wherein the diluent is water, a buffer, a buffered salt solution or a salt solution.

18. The pharmaceutical composition according to claim 15, wherein the carrier is selected from the group consisting of cocoa butter and vitebesole.

19. A method of treating a disease or condition in a subject in need thereof, which disease or condition can be affected by an increase of cyclic adenosine monophosphoric acid contents in the brain of the subject, comprising administering to the subject the blood serum according to claim 14 in an amount effective to treat the disease or condition.

20. A method of improving the cognitive skill, learning skill, or a combination thereof, in a subject in need thereof, comprising administering to the subject the blood serum according to claim 14 in an amount effective to improve the cognitive skill learning skill or a combination thereof in the subject.

21. A method of treating seizure in a subject comprising administering to the subject the blood serum according to claim 14 in an amount effective to treat the seizure in the subject.

22. A method of treating a nervous disease in a subject comprising administering the blood serum according to claim 14 in an amount effective to treat said nervous disease in the subject.

23. A method of treating a proliferative disease or apoplexy in a subject, comprising administering the blood serum according to claim 14 in an amount effective to treat the proliferative disease or apoplexy in the subject.

24. The method according to claim 23, wherein the proliferative disease is selected from the group consisting of malignomas of the gastrointestinal or colorectal tract, the liver, the pancreas, the kidney, the bladder, the thyroid, the prostate, the endometrium, the cervix, the ovary, the uterus, the testes, the skin, the oral cavity; melanoma; dysplastic oral mucosa; invasive oral cancers; small cell and non-small cell lung carcinomas; mammary tumors transitional and squamous cell cancers; neurological malignancies. soft tissue sarcomas; hemangioamas and endocrinological tumors.

25. The method according to claim 23, wherein the proliferative disease comprises cells similar to the human T cell lymphoma cell line Jurkat, the human B cell lymphoma cell line Raj, the human melanoma cell line Bro, the human cervical cancer cell line HeLa, the human adenocarcinoma cell line MCF-7, the osteosarcoma cell line Mg63, the fibrosarcoma cell line HT1080, the neuroblastoma cell line IMR-32 and the hepatocarcinoma cell line HepG2.

26. The method of claim 19 wherein the blood serum is administered to the subject in an amount ranging from 50 to 150 mg/kg body weight.
